(19) United States
(12) Patent Application Publication
(10) Pub. No.: US 2023/0364437 A1
(43) Pub. Date: Nov. 16, 2023

(54) BEAUTY DEVICE

(71) Applicants: Jcraft Co., Ltd., Osaka (JP); BELEGA Co., Ltd., Osaka (JP)

(72) Inventor: Hiroshi UENO, Osaka (JP)

(21) Appl. No.: 18/029,707

(22) PCT Filed: Oct. 1, 2021
(86) PCT No.: PCT/JP2021/036475

§ 371 Date: Mar. 31, 2023

(30) Foreign Application Priority Data
Oct. 16, 2020 (JP) 2020-174670

(51) Int. Cl. A61N 1/40

(52) U.S. Cl. CPC A61N 1/403

(57) ABSTRACT

A beauty device applies electric currents of different frequencies to a human body through electrodes so as to interfere with each other includes at least three electrodes for sending the electric currents of the frequencies of 60 kHz to 65 kHz with frequency differences between the frequencies of 100 Hz to 1000 Hz, and the electrodes includes more than a pair of negative electrodes and positive electrodes. The electric currents of the frequencies of 60 kHz to 65 kHz reach the area of a fat layer and a muscle layer under the skin and generate heat, and the electric currents of the interference waves of 100 Hz to 1000 Hz allow to move muscle in the area of the fat layer and the muscle layer under the skin, by utilizing the action of difference between contraction and relaxation of the muscle, which is unique to low-frequency waves.

### TECHNICAL FIELD

The present invention relates to a beauty device configured not only to make a fat layer and a muscle layer vibrate, but also to apply heat.

### BACKGROUND ART

The present applicant has proposed an interference frequency slimming device which is configured to perform appropriate slimming massage even at home by applying frequency currents with predetermined differences to a plurality of pairs of electrodes, in Patent Document 1 (Japanese Patent Application Laid-Open Publication No. 2007-125300).

Patent Document 1 discloses the solution of the problems in which although a slimming device forcibly exercises muscle (and fat) under the skin by applying an electric current of a specific frequency, that is, it exhibits a slimming effect, a low-frequency current flows through epidermis and thus causes a pain sensation, while a medium-frequency current flows under the skin, and does not make the body feel the slimming effect on a muscle layer in spite that the current causes a moderate pain sensation.

The slimming device in Patent Document 1 generates a low-frequency wave which does not cause a pain sensation as a whole waveform, by interfering the medium-frequency waves reaching a muscle layer with each other by amplitude modulation, resulting in that the slimming device does not cause a pain sensation, that is, it makes the body surely feel the movement of muscle under the skin even by applying a medium-frequency current.

The slimming device in Patent Document 1 exhibits the slimming effect just by applying electrical stimulation to vibrate muscle, and the degree of improvement in slimming effect reaches the limit. The present applicant has realized that not only the application of electrical vibration to muscle but also the application of heat may eliminate the present limit.

However, the method of simply applying heat (warming) from the epidermis does not allow the heat of a low temperature to penetrate into a fat layer or a muscle layer in the case of a thick subcutaneous fat. If the temperature is increased so that the heat penetrates into the fat layer and the muscle layer, the heat is felt just at the epidermis, resulting in failure of effective application of heat.

### CITATION LIST

### Patent Literature

[Patent Document 1] Japanese Patent Application Laid-Open Publication No. 2007-125300

### SUMMARY OF INVENTION

### Technical Problem

The problem to be solved by the present invention is that it is difficult to effectively apply heat by the method of applying heat in addition to vibrating a fat layer and a muscle layer in order to improve a slimming effect.

In order to solve the problem described above, in the present invention, a beauty device which is configured to apply electric currents of different frequencies to a human body through electrodes so as to interfere with each other includes at least three electrodes for sending the electric currents of the frequencies of 60 kHz to 65 kHz with frequency differences therebetween of 100 Hz to 1000 Hz, and the electrodes include more than a pair of negative electrodes and positive electrodes.

### Advantageous Effects of Invention

According to the present invention, the following effect is exhibited. A current of a frequency of 60 kHz to 65 kHz reaches the area of a fat layer and a muscle layer under the skin, and generates heat. A current of an interference wave of 100 Hz to 1000 Hz generated due to the frequency difference between the frequencies of 60 kHz to 65 kHz reaches the area of a fat layer and a muscle layer under the skin, and allows to move muscle, by utilizing the difference in action between contraction and relaxation of the muscle, which is unique to low-frequency waves. The effect provides a tightening muscle effect and a burning fat effect, resulting in providing a higher sliming effect.

### BRIEF DESCRIPTION OF DRAWINGS

FIG. 1 illustrates a diagram for explaining the beauty device according to the present invention.

FIG. 2a illustrates a conceptual diagram for explaining an operation state of the beauty device according to the present invention, and FIG. 2b illustrates a conceptual diagram for explaining an interference wave.

FIG. 3 illustrates the results of the experiment conducted to verify the effect of the beauty device according to the present invention.

FIG. 4 illustrates the results of the experiment conducted to verify the effect of the beauty device according to the present invention.

### DESCRIPTION OF EMBODIMENTS

The object of the present invention is not only to vibrate a fat layer and a muscle layer but also to apply heat. The object is realized through the configuration which includes at least three electrodes for sending electric currents of frequencies of 60 kHz to 65 kHz with the frequency differences therebetween of 100 Hz to 1000 Hz, and in which the electrodes include more than a pair of negative electrodes and positive electrodes.

A high frequency higher than 65 kHz does not generate moderate heat (for example, 40° C. to 42° C.) without increasing the output (current value). Thus, this type of device if using such a high frequency band provides a poor balance of heat generation to the output. The amount of heat generated by such a high frequency higher than 65 kHz is more than a standard level, and may cause an error in usage or circuit control, possibly resulting in destroying internal tissues of the human body or causing burns, and further damaging a home use wireless LAN or a communication device, as an example, due to the interference with such devices, or the own device. On the other hand, a high frequency lower than 60 kHz not necessarily requiring a high output, unlike the case described above, has insufficient penetrability into deep subcutaneous tissues, and may generate heat only in the fat layer having a high resistance value.

The frequencies of 60 kHz to 65 kHz with frequency differences therebetween lower than 100 Hz make the body feel the movements of fat and muscle, and cause a pain sensation, so that the pain is transmitted to subcutaneous nerves. On the other hand, the frequencies of 60 kHz to 65 kHz with frequency differences therebetween higher than 1000 Hz cause a pain sensation relieved in some degree, and does not make the body feel the movement of fat or muscle.

### EXAMPLES

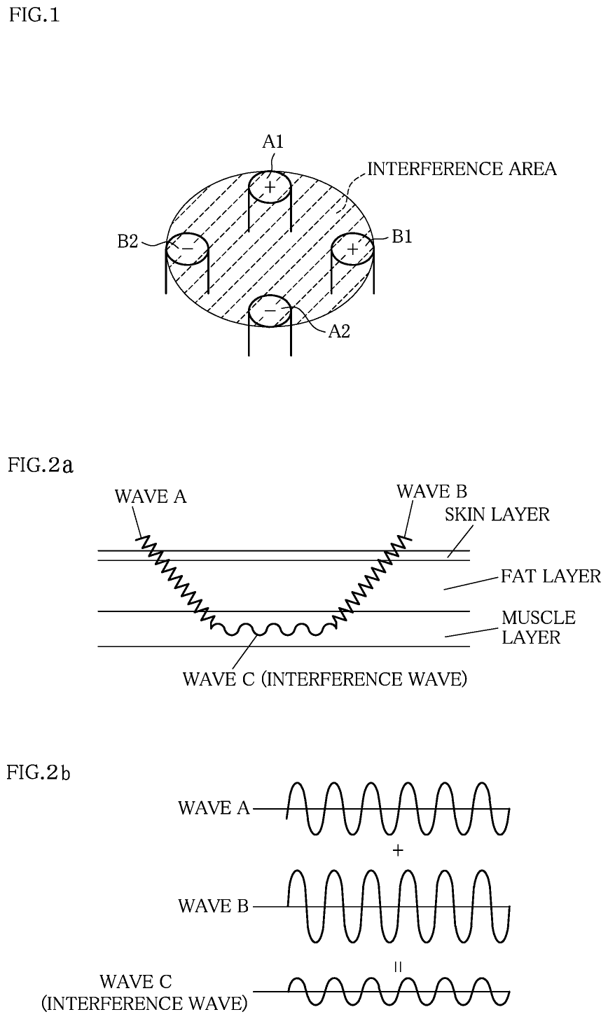

The results of the experiment conducted to verify the effect of the beauty device according to the present invention will be described below. The conditions of the experiment are as follows. As shown in FIG. 1, four electrodes of a positive electrode A1, a negative electrode A2, a positive electrode B1, and a negative electrode B2 are disposed alternately adjacent to each other to form two pairs of electrodes. In this configuration, a current of 15 mA of a reference frequency (wave A) is sent from one side (electrode A), and a current of 15 mA of a frequency (wave B) higher than the frequency of the wave A is sent from the other side (electrode B). It is assumed that the interference area of the wave A and the wave B under the skin is approx. 10 cm². The output voltage of the wave A and the wave B is set to 30 V at a load of 1 KΩ, and a current flowing time is set to three minutes.

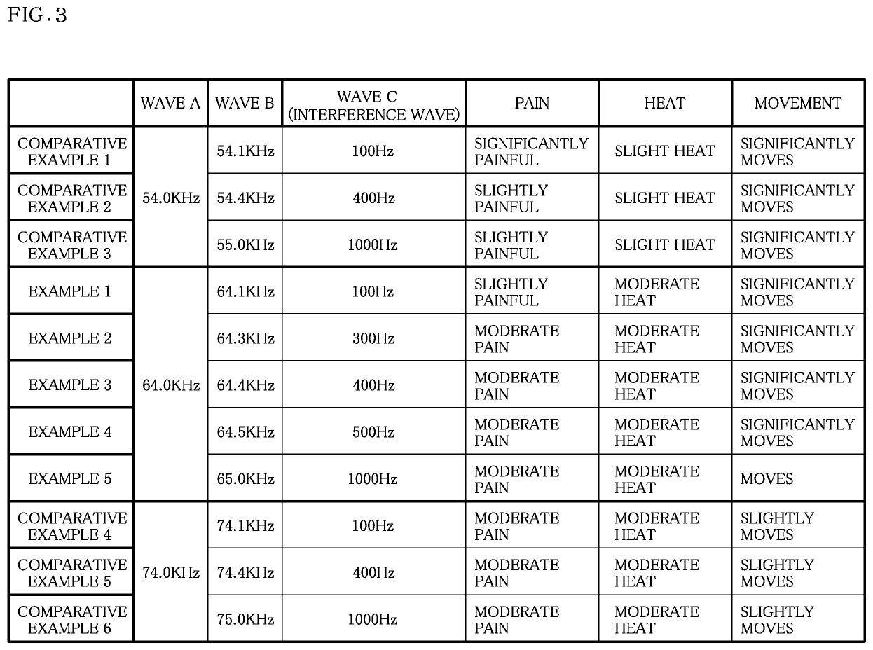

FIG. 2 illustrates the image of the experiment. In the experiment described above, 50 participants evaluated physical sensations of pain, heat sensation, and movement of muscle caused by an interference wave C of the wave A and the wave B in the interference area under the skin. FIG. 3 illustrates the results of the experiment.

According to the results of the experiment, overall, the interference wave C of a frequency closer to 100 Hz makes the body more strongly feel the movements of muscle and fat, while the interference wave C of a frequency closer to 1000 Hz makes the body more weakly feel the movements of muscle and fat. The wave C of a frequency lower than 100 Hz causes a strong pain sensation, while the wave C of a frequency higher than 1000 Hz does not make the body feel the movement of muscle or fat, which are not in the table.

The frequency band of the 54 kHz range of the wave A and the wave B does not make the body feel heat as a whole, or makes the body feel heat, if any, only slightly in the latter half of the measurement time. The frequency band of the 74 kHz range does not make the body feel the movements of muscle and fat.

The results of the physical sensation with respect to Comparative Examples 1 to 6 are as follows.

The Comparative Example 1 in which the wave C of 100 Hz is generated with the wave A of 54.0 kHz set as a fixed wave and the wave B set to 54.1 kHz causes a stabbing pain sensation and makes the body feel slight heat, and further makes the body feel the movements of muscle and fat.

The Comparative Example 2 in which the wave C of 400 Hz is generated with the wave A set to 54.0 kHz as a fixed wave and the wave B set to 54.4 kHz causes a pain sensation relieved in some degree compared to the stabbing pain sensation of the Comparative Example 1 and makes the body feel slight heat, and further makes the body feel the movements of muscle and fat.

The Comparative Example 3 in which the wave C of 1000 Hz is generated with the wave A set to 54.0 kHz as a fixed wave and the wave B set to 55.0 kHz causes a pain sensation in some degree and makes the body feel slight heat, and makes the body feel the movements of muscle or fat.

With the wave A set to 74.0 kHz as a fixed wave, all of the Comparative Example 4 in which the wave C of 100 Hz is generated with the wave A and the wave B set to 74.1 kHz, the Comparative Example 5 in which the wave C of 400 Hz is generated with the wave A and the wave B set to 74.4 kHz, and the Comparative Example 6 in which the wave C of 1000 Hz is generated with the wave A and the wave B set to 75.0 kHz cause a moderate pain sensation and makes the body feel heat, and further makes the body slightly feel the movements of muscle and fat.

The results of the physical sensation with respect to Examples 1 to 5, unlike the Comparative Examples 1 to 6, are as follows.

The Example 1 in which the wave C of 100 Hz is generated with the wave A set to 64.0 kHz as a fixed wave and the wave B set to 64.1 kHz causes a pain sensation in some degree and makes the body feel sufficient and moderate heat, and further makes the body feel the movements of muscle and fat.

With the wave A set to 64.0 kHz as a fixed wave, all of the Example 2 in which the wave C of 300 Hz is generated with the wave A and the wave B set to 64.3 kHz, the Example 3 in which the wave C of 400 Hz is generated with the wave A and the wave B set to 64.4 kHz, and the Example 4 in which the wave C of 500 Hz is generated with the wave A and the wave B set to 64.5 kHz cause a moderate pain sensation and make the body feel moderate heat, and further make the body feel the movements of muscle and fat.

The Example 5 in which the wave C of 1000 Hz is generated with the wave A set to 64.0 kHz as a fixed wave and the wave B set to 65.0 kHz causes a moderate pain sensation and makes the body feel moderate heat, and further makes the body feel the movements of muscle and fat more weakly compared to the Examples 1 to 4, but clearly.

The following experiment is conducted to quantify “heat” among the so-called sensory tests described above. The conditions of the experiment are that the four electrodes of the positive electrode A1, the negative electrode A2, the positive electrode B1, and the negative electrode B2 are disposed alternately adjacent to each other to form two pairs of electrodes, and an electric current of the wave A and the wave B at 30 V is applied for three minutes as in the experiment described above (the Examples 1 to 5 and the Comparative Examples 1 to 6).

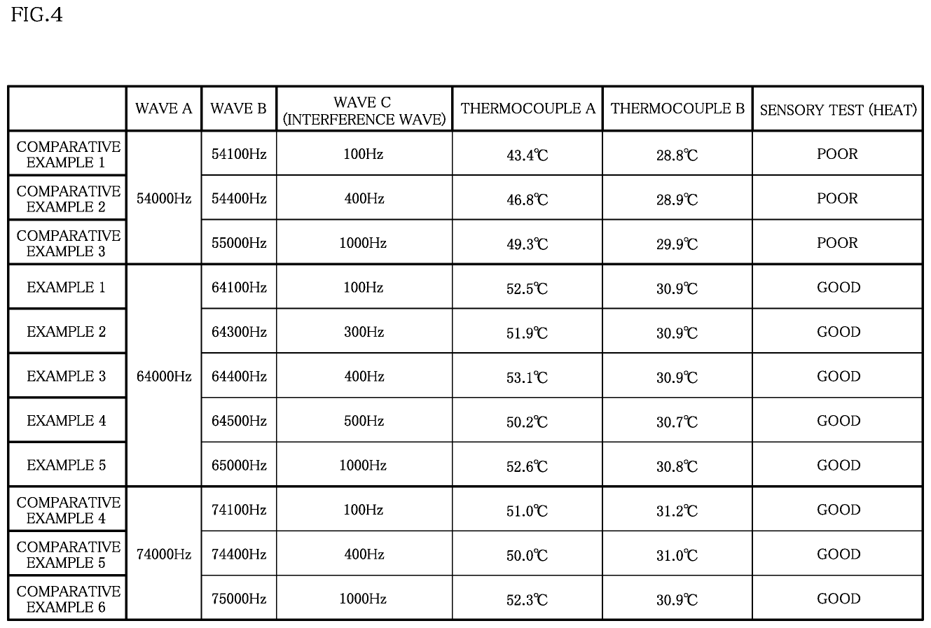

A thermocouple A is arranged in an interference area, and a thermocouple B is arranged at a distance from the position where the thermocouple A is arranged (free from electric current). The temperatures after the application of the electric current for three minutes are measured by these thermocouples A, B. FIG. 4 illustrates the results.

According to the results of the experiment, overall, although it is difficult to grasp the trend in the results with variations with respect to the interference wave C of 100 Hz and 400 Hz, the interference wave C of 1000 Hz obviously increases the temperatures of the deep subcutaneous tissues where the interference wave C reaches, compared to the case of 100 Hz and the case of 400 Hz.

With respect to the frequency band of the 54 kHz range of the wave A and the wave B, the temperatures measured by the thermocouple A are lower than 50.0° C. The results are consistent with the results in which, in the sensory test, the frequency band does not make the body feel heat as a whole, or makes the body feel heat, if any, only slightly in the latter half of the measurement time. With respect to the frequency bands of the 64 kHz range and the 74 kHz range, the temperatures measured by the thermocouple A are higher than 50.0° C. The results are consistent with the results in which, in the sensory test, the frequency bands make the body feel moderate heat.

The results in which the Comparative Examples 1 to 3 do not generate heat both in substantial numerical values and in physical sensation reveal that none of the Comparative Examples 1 to 3 is able to apply heat. On the other hand, although the Comparative Examples 4 to 6 are able to apply heat both in substantial numerical values and in physical sensation, the Comparative Examples 4 to 6 cause the malfunctions in physical sensation which are not appeared in FIG. 4 and will be described below.

It is estimated that, with respect to the Comparative Examples 4 to 6 in which the wave A is set as a fixed wave to the 74 kHz range in the first place, the interference waves (wave C) are generated in deeper subcutaneous tissues compared to the Examples 1 to 5 of the 64 kHz range. Accordingly, even though the measured temperatures are in the same 50° C. range, the interference waves make the body feel moderate heat for three minutes of the electric current flowing, and the heat is stored for a long period in the deep subcutaneous tissues, resulting in providing physical discomfort.

As described above, the present invention allows to effectively apply electrical stimulation to and generate heat in the fat layer and the muscle layer under the skin, and is thus expected to exhibit more slimming effect compared to the device configured to apply only heat using a specific frequency band, or the device configured to apply electrical stimulation for vibration only.

### REFERENCE SIGNS LIST

A1, B1: POSITIVE ELECTRODE
A2, B2: NEGATIVE ELECTRODE

1. A beauty device configured to apply electric currents of different frequencies to a human body through electrodes so as to interfere with each other, the beauty device comprising
at least three electrodes for sending the electric currents of the frequencies of 60 kHz to 65 kHz with frequency differences between the frequencies of 100 Hz to 1000 Hz, the electrodes including more than a pair of negative electrodes and positive electrodes.
